(19) United States
(12) Patent Application Publication
(10) Pub. No.: US 2025/0248707 A1
(43) Pub. Date: Aug. 7, 2025

(54) CLIP APPLIER WITH STABILIZING MEMBER

(71) Applicant: TELEFLEX MEDICAL INCORPORATED, Morrisville, NC (US)

(72) Inventor: Salvatore CASTRO, Raleigh, NC (US)

(21) Appl. No.: 19/184,363

(22) Filed: Apr. 21, 2025

Related U.S. Application Data
(63) Continuation of application No. 17/213,524, filed on Mar. 26, 2021, now Pat. No. 12,279,774.
Application No. 17/213,524 is a continuation of application No. PCT/US2019/053145, filed on Sep. 26, 2019.
(60) Provisional application No. 62/737,043, filed on Sep. 26, 2018.

(51) Int. Cl. A61B 17/10; A61B 17/00; A61B 17/122; A61B 17/128

(52) U.S. Cl. CPC A61B 17/10; A61B 17/122; A61B 17/128; A61B 17/1285; A61B 2017/00367

(57) ABSTRACT

A clip applier may be configured to apply a surgical clip. The clip applier may include first and second jaw members configured to engage the surgical clip, an actuating member configured to pivot at least one of the first and second jaw members between an open configuration and a closed configuration, and a stabilizing member configured to engage the surgical clip. The stabilizing member may be configured to move longitudinally with respect to the clip applier from a distal position at least partially between the first and second jaw members to a proximal position at least partially between the first and second jaw members. Movement of the stabilizing member between the distal position and the proximal position may be actuated by at least one of the first jaw member, the second jaw member, and the actuating member.

### PRIORITY

This application is a continuation of U.S. patent application Ser. No. 17/213,524 (filed Mar. 26, 2021), which is a continuation of International Patent Application PCT/US2019/053145 (filed Sep. 26, 2019), which claims the benefit of priority of U.S. Provisional Patent Application No. 62/737,043 (filed Sep. 26, 2018 and now expired), the disclosures of which are expressly incorporated herein by reference in their entirety.

### TECHNICAL FIELD

The present disclosure relates generally to clip appliers, and more particularly, to clip appliers with a stabilizing member configured to stabilize a surgical clip.

### BACKGROUND

Ligation of tissue (e.g., blood vessels, lymph nodes, nerves, fallopian tubes, and cardiac tissue) is a common practice for many surgical procedures. This can be performed by closing the vessel with a surgical clip or by suturing the vessel with the surgical thread. The use of surgical thread requires complex manipulations of a needle and surgical thread to form knots required to secure the vessel. Such complex manipulations are time consuming and difficult to perform, particularly in endoscopic surgical procedures characterized by limited space and/or visibility. In contrast, surgical clips are relatively quick and easy to apply. Accordingly, the use of surgical clips in endoscopic and open surgical procedures has grown dramatically.

### SUMMARY

The present inventor recognizes that there is a need to improve one or more features of the clip appliers, such as stability of the surgical clip in a clip applier. Surgical clips are often applied by clip appliers with a pair of opposing jaws. Currently available clip appliers often secure the clip with two points of contact, for example, the opposing jaws may engage bosses on distal ends of the surgical clip. However, the two points of contact do not provide sufficient stability to the surgical clip, which may cause the surgical clip to become misaligned relative to the clip applier during a surgical procedure, or even fall out. The disclosed methods and systems are directed to mitigating or overcoming one or more of the problems set forth above and/or other problems in the prior art.

A first aspect of the present invention is directed to a clip applier configured to apply a surgical clip to tissue. The clip applier may include first and second jaw members configured to engage the surgical clip, an actuating member configured to pivot at least one of the first and second jaw members between an open configuration and a closed configuration, and a stabilizing member configured to engage the surgical clip. The stabilizing member may be configured to move longitudinally with respect to the clip applier from a distal position at least partially between the first and second jaw members to a proximal position at least partially between the first and second jaw members. Movement of the stabilizing member between the distal position and the proximal position may be actuated by at least one of the first jaw member, the second jaw member, and the actuating member.

In some embodiments, the stabilizing member may be configured to disengage the surgical clip when the stabilizing member is in the proximal position. In some embodiments, the stabilizing member may have at least one slot, and at least one of the first and second jaw members may have a pin received in a slot of the at least one slot, where the pin slides along the slot to actuate the stabilizing member. In some embodiments, the at least one slot may be angled relative to a longitudinal axis of the clip applier. In some embodiments, the at least one slot may include a first slot and a second slot, and each of the first and second jaw members may include a pin. In some embodiments, the actuating member may be directly attached to a proximal portion of the stabilizing member. In some embodiments, the stabilizing member may be configured to stabilize a proximal portion of the surgical clip in a lateral direction. In some embodiments, the stabilizing member may include first and second walls or protrusions on a distal portion configured to receive the proximal portion of the surgical clip therebetween. In some embodiments, the first jaw member may have a first inner channel, and the second jaw member may have a second inner channel, where the stabilizing member may be received in the first and second inner channels in the closed configuration. In some embodiments, the clip applier may have a linkage connecting the actuating member and the first and second jaw members. In some embodiments, the actuating member may include an actuating rod. In some embodiments, the first jaw member may be configured to engage a distal portion of a first leg member of the surgical clip, the second jaw member may be configured to engage a distal portion of a second leg member of the surgical clip, and the stabilizing member may be configured to engage a proximal portion of the surgical clip. In some embodiments, the stabilizing member is constrained to longitudinal movement between the distal and proximal positions. In some embodiments, actuating the actuating member, pivoting the at least one of the first and second jaw members to the closed configuration, and moving the stabilizing member from the distal position to the proximal position are performed simultaneously.

A second aspect of the present invention is directed to a method of applying a surgical clip with a clip applier. The method may include receiving the surgical clip between first and second jaw members of the clip applier, and engaging the proximal portion of the surgical clip with a stabilizing member in a distal position at least partially between the first and second jaw members. The method may further include moving an actuating member to pivot at least one of the first and second jaws members toward a closed configuration to close the surgical clip, and moving the stabilizing member from the distal position to a proximal position by actuation of at least one of the first jaw member, the second jaw member, and the actuating member.

In some embodiments, the method may further include disengaging the surgical clip from the stabilizing member when the stabilizing member is in the proximal position. In some embodiments, the method may further include sliding a pin of at least one of the first and second jaw members through a slot in the stabilizing member to actuate the stabilizing member. In some embodiments, moving the stabilizing member may be actuated through a direct connection with the actuating rod. In some embodiments, the method may further include receiving the proximal portion of the surgical clip between first and second walls of the stabilizing member to stabilize the proximal portion of the surgical clip. In some embodiments, the method may further include actuating a linkage with the actuating member to pivot at least one of the first and second jaw members. In some embodiments, the method may further include receiving the stabilizing member in a first inner channel of the first jaw member and a second inner channel of the second jaw member in the closed configuration. In some embodiments, the method may further include engaging a distal portion of a first leg member with the first jaw member and a distal portion of a second leg member with the second jaw member. In some embodiments, moving the stabilizing member is constrained to longitudinal movement between the distal and proximal positions. In some embodiments, moving the actuating member, pivoting the at least one of the first and second jaw members to the closed configuration, and moving the stabilizing member from the distal position to the proximal position are performed simultaneously.

### BRIEF DESCRIPTION OF THE DRAWINGS

In order that the disclosure may be readily understood, aspects of this disclosure are illustrated by way of examples in the accompanying drawings.

FIG. 1 illustrates a manual clip applier of the present disclosure.

FIG. 2 illustrates a first perspective view of a first exemplary embodiment of a distal end effector of the manual clip applier of FIG. 1.

FIG. 3 illustrates a second perspective view of the first exemplary embodiment of FIG. 2.

FIG. 4 illustrates a side view of the first exemplary embodiment of FIGS. 2-3 in an open configuration with a surgical clip.

FIG. 5 illustrates a side view of the first exemplary embodiment of FIGS. 2-4 in a closed configuration with the surgical clip.

FIG. 6 illustrates a side view of a second exemplary embodiment of a distal end effector of the manual clip applier of FIG. 1 in an open configuration with a surgical clip.

FIG. 7 illustrates a side view of the second exemplary embodiment of FIG. 6 in a closed configuration with the surgical clip.

The same or similar reference numbers are used in the drawings and the following detailed description to refer to the same or similar parts.

### DETAILED DESCRIPTION

The invention will now be described with reference to the figures, in which like reference numerals refer to like parts throughout. In accordance with conventional practice, as used herein, and unless otherwise indicated herein, the term “proximal” refers to the relative positioning of a device or its component generally closer to the medical personnel handling or manipulating the device as it is intended to be used, and the term “distal” refers to the relative positioning of a device or its component further from the medical personnel handling or manipulating the device as it is intended to be used. The term “vertical” with reference to the clip applier of a component refers to a relative direction of the clip applier parallel or along a plane extending evenly through both jaw members or similarly to the component. The term “longitudinal” relative to the clip applier or a component refers to a relative direction along a long axis or length of the clip applier or the component. The term “lateral” relative to the clip applier or a component refers to a relative direction parallel or along a plane extending perpendicularly between the first and second jaw members or similarly to the component.

The present invention is generally directed to a manual clip applier configured to increase stability of surgical clips during a medical procedure. The manual clip applier may include a stabilizing member disposed between first and second jaw members. The stabilizing member and the first and second jaw members may provide at least three points of contact with the surgical clip to prevent relative movement of the surgical clip during the medical procedure. The stabilizing member may have vertical walls extending from a distal portion configured to receive a proximal portion of the surgical clip and laterally stabilize the surgical clip. The vertical walls may extend from a distal portion of the stabilizing member on opposing sides to reduce lateral movement of the surgical clip. The vertical walls may stabilize the surgical clip while the surgical clip is loaded, manipulated, and/or delivered to tissue (e.g., to ligate a blood vessel), preventing the surgical clip from fish-tailing. The stabilizing member may further have lateral protrusions extending between the vertical walls and being configured to reduce vertical movement.

The stabilizing member may be configured to move longitudinally between a first, distal position and a second, proximal position to allow the stabilizing member to apply a sufficient distal stabilizing force when the surgical clip is received between the first and second jaw members of the clip applier during front-loading of the manual surgical clip from a clip cartridge. The stabilizing member may longitudinally retract to the proximal position during compression of the surgical clip, so the stabilizing member does not interfere with elongation of the surgical clip during compression and/or release of the surgical clip after being closed/latched onto the tissue. The movement of the stabilizing member may be directly actuated by at least one of the first jaw member, the second jaw member, and an actuating member (e.g., an actuating rod) that actuates the first and second jaw members. Thus, the stabilizing member is actuated independently of the surgical clip, such that the stabilizing member moves longitudinally when the clip applier is not loaded and in the absence of a surgical clip. The actuation of the stabilizing member may be based on movement of at least one of the first jaw member, the second jaw member, and the actuating member, such that the stabilizing member moves between proximal and distal positions as the first and second jaw members open and close. For example, in some embodiments, the stabilizing member may include angled slots that receive pins from the first and second jaw members. The angled slots may directly cause the stabilizing member to retract while the jaw members pivot closed and the stabilizing member to advance while the jaw members pivot open. In some embodiments, a distal end of the actuating member may be directly connected to a proximal end of the stabilizing member, such that retraction of the actuating member directly causes the stabilizing member to retract as the jaw members close and advancement of the actuating member directly causes the stabilizing member to advance as the jaw members open. In some embodiments, a distal end of the actuating member may be integrally attached to a proximal end of the stabilizing member. Thus, moving/retracting the actuating member, pivoting at least one of the first and second jaw members toward a closed configuration, and moving the stabilizing member from the distal position to the proximal position may be performed simultaneously in the absence of a surgical clip. The opposite movements may be performed simultaneously as the first and second jaw members pivot toward an open configuration, for example, to load a second surgical clip.

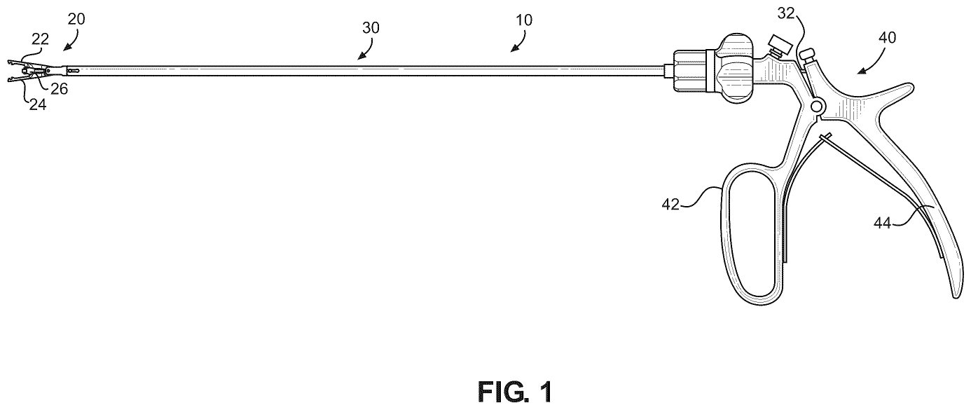

FIG. 1 illustrates a manual clip applier 10 according to the present disclosure. The clip applier 10 may include a distal end effector including a jaw mechanism 20 at a distal end of an elongated shaft 30 and a handle mechanism 40 at a proximal end of the elongated shaft 30. The jaw mechanism 20 may include a first jaw member 22, a second jaw member 24. and a stabilizing member 26 between the first and second jaw members 22, 24. The jaw mechanism 20 may be actuated by the handle mechanism 40 via an actuating member 32 extending through the elongated shaft 30. The handle mechanism 40 may include a first handle member 42 and a second handle member 44. For example, a proximal end of the elongated shaft 30 may be attached to the first handle member 42, and a proximal end of the actuating member 32 may be attached to the second handle member 44, such that relative movement or pivoting of the handle members 42, 44 may cause relative movement of the actuating member 32 to actuate the jaw mechanism 20. In some embodiments, the jaw mechanism 20 may be releasably attached to the handle mechanism 40 and the elongated shaft 30 for cleaning purposes. For example, a pin on the distal end effector may be received in a slot on the distal end of the elongated shaft 30. The actuation of the first jaw member 22, the second jaw member 24, and the stabilizing member 26 is discussed in the exemplary embodiments herein.

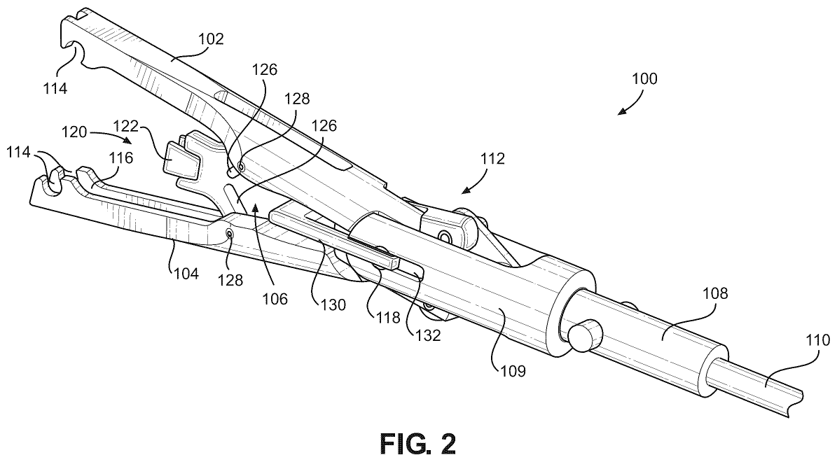

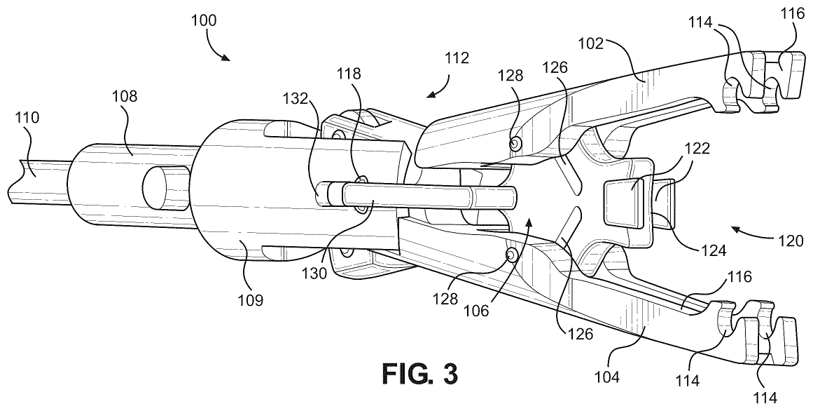

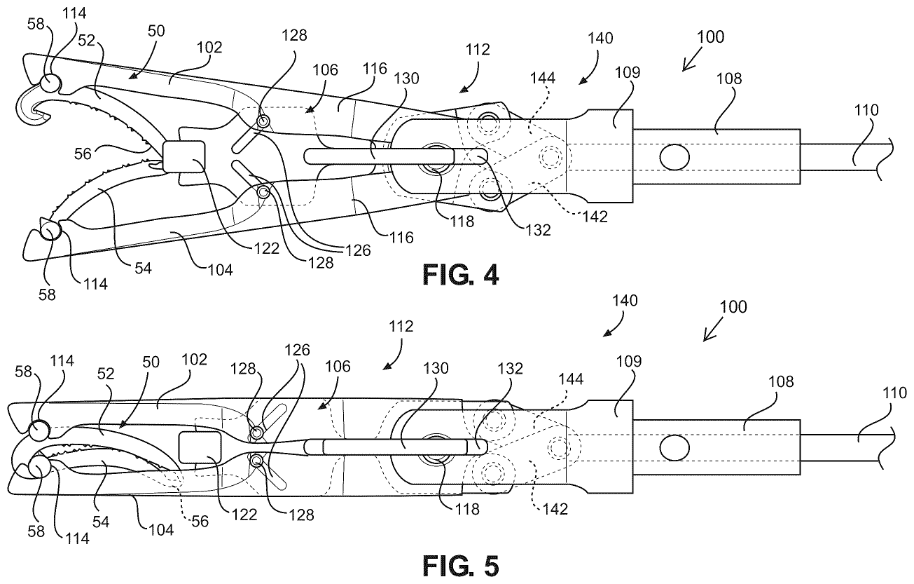

FIGS. 2-5 illustrate a first embodiment 100 of the distal end effector of the manual clip applier 10 of FIG. 1. The clip applier 100 may be configured to apply a surgical clip 50 (as illustrated in FIGS. 4-5). The clip applier 100 may include a first jaw member 102, a second jaw member 104, a stabilizing member 106, a shaft 108, and a handle mechanism (as illustrated in FIG. 1). Actuation of the handle mechanism may retract and/or advance an actuating member (e.g., an actuating rod) 110 through the shaft 108 to cause the first and second jaw members 102, 104 to pivot between an open configuration (e.g., FIG. 4) and a closed configuration (e.g., FIG. 5). The first and second jaw members 102, 104 may thus compress the surgical clip 50 by pivoting first and second leg members 52. 54 about a hinge portion 56.

The first and second jaw members 102, 104 may be pivotally coupled at a hinge mechanism 112 having a pivot pin 118 on a distal portion 109 of the shaft 108. The first and second jaw members 102, 104 may receive the surgical clip 50 between the first and second jaw members 102, 104, and the first and second jaw members 102, 104 may stabilize the surgical clip 50 at points of contact on distal portions of the first and second leg members 52, 54. For example, as illustrated in FIGS. 2-5, the first and second jaw members 102, 104 may have distal portions with one or more recesses 114 that receive one or more bosses 58 on distal portions of the first and second leg members 52, 54. Each of the first and second jaw members 102, 104 may further include a longitudinal channel 116 configured to receive a portion of the surgical clip 50 and/or the stabilizing member 106.

The stabilizing member 106 may be received between the first and second jaw members 102, 104 to provide additional stability to the surgical clip 50. The stabilizing member 106 may include an elongated body having a distal portion 120 that receives and/or engages the proximal portion (e.g., the hinge portion 56) of the surgical clip 50. The distal portion 120 may include vertical walls or protrusions 122 extending from the distal portion 120 of the elongated body on opposing sides of the stabilizing member 106. For example, the vertical walls 122 may be integrated, welded, and/or secured to opposing lateral sides of the elongated body of the stability member 106, such that the width defined by the vertical walls 122 may be wider than the width of the remaining length of the stabilizing member 106. The vertical walls 122 may define a channel 124 therebetween configured to receive the proximal portion (e.g., the hinge portion 56) of the surgical clip 50, reducing lateral movement of the surgical clip 50. The vertical walls 122 may be substantially parallel, and may not extend the entire height of the distal portion 120. The distal portion 120 may further include lateral protrusions (not shown) extending laterally between the vertical walls 122 to engage the proximal portion of the surgical clip 50. Thus, the distal portion 120 may receive, grip, and/or stabilize the surgical clip 50 in a lateral and/or vertical direction when positioned between the first and second jaw members 102, 104. Embodiments of the distal portion 120 of the stabilizing member 106 are further disclosed in U.S. Pat. Pub. No. 2018/0271534, the disclosure of which is expressly incorporated herein in its entirety.

The stabilizing member 106 may be positioned symmetrically between the first and second jaw members 102, 104. The positioning of the stabilizing member 106 may allow a user to pick up the surgical clip 50 from a clip cartridge (not shown) with the clip applier 100 in either of two opposite orientations. For example, the first jaw member 102 may engage either of the first leg member 52 or the second leg member 54 of the surgical clip 50, while the second jaw member 104 engages the other of the first leg member 52 and the second leg member 54. The three point engagement between the clip applier 100 and the surgical clip 50 increases the security of the surgical clip 50. Two points of contact occur on the surgical clip 50 at distal surfaces of the first and second leg members 52, 54, and a third point of contact occurs at a proximal portion of the surgical clip 50 (e.g., the hinge portion 56). The surgical clip 50 may remain positively engaged between the first and second jaw members 102, 104, despite external forces.

As illustrated in FIGS. 4-5, the first and second jaw members 102, 104 may be configured to compress the surgical clip 50 by applying opposing forces on the first and second leg members 52. 54. The clip applier 100 may be initially loaded with the surgical clip 50 from the clip cartridge (not shown), such that the first and second jaw members 102, 104 engage distal portions of the leg members 52, 54 (e.g., bosses on surgical clip engaged by recesses 114) and the hinge portion 56 is received between the vertical walls 122 of the stabilizing member 106 when the stabilizing member 106 is in a distal position. Actuation of the handle mechanism may retract the actuating member 110, which may be coupled to the first and second jaw member 102, 104 through a linkage 140 received in the distal portion 109 of the shaft 108. For example, a first link 142 may have a first end pivotally coupled to the actuating member 110 and a second end pivotally coupled to a proximal end of the first jaw member 102. A second link 144 may have a first end pivotally coupled to the actuating member 110 and a second end pivotally coupled to a proximal end of the second jaw member 104. The attachment of the linkage 140 may be proximal of the hinge mechanism 112, such that retraction of the actuating member 110 causes the first and second jaw members 102, 104 to pivot to a closed configuration. The distal portion 109 may be open and enlarged relative to a proximal portion of the shaft 108, such that the distal portion 109 may receive proximal ends of the jaw members 102, 104 that are pivoted about the pivot pin 118.

As the first and second jaw members 102, 104 pivot, the stabilizing member 106 may be configured to move longitudinally between a first, distal position at least partially between the first and second jaw members 102, 104 (e.g., FIG. 4) and a second, proximal position at least partially between the first and second jaw members 102, 104 (e.g., FIG. 5). The stabilizing member 106 may be constrained to longitudinal movement between the first and second positions. The elongated body of the stabilizing member 106 may be received in the longitudinal channels 116 of the first and/or second jaw members 102, 104 in the first and/or second positions to prevent impeding of the pivoting of the jaw members 102, 104. After compression of the surgical clip 50, the jaw members 102, 104 may pivot to the open configuration thus returning stabilizing member 106 to the first, distal position to engage a second surgical clip 50 from a cartridge.

As illustrated in the FIGS. 4-5, the movement of the stabilizing member 106 may be directly actuated by the pivoting of the jaw members 102, 104. For example, the stabilizing member 106 may include one or more slots 126 slideably receiving a pin 128 of one or more the jaw members 102, 104. Thus, the pins 128 may be in a proximal position in the slots 126 when the jaw members 102, 104 are in an open configuration and the stabilizing member 106 is in the distal position to engage the surgical clip 50 (e.g., FIG. 4). As the jaw members 102, 104 pivot to the closed position, the pins 128 may slide through the slots 126 to a distal position when the jaw members 102, 104 are in a closed configuration and the stabilizing member 106 is in the proximal position to disengage the surgical clip 50 (e.g., FIG. 5). As further illustrated in FIGS. 4-5, stabilizing member 106 may include first and second slots 126 to receive a pin 128 from each of the first and second jaw members 102, 104 to allow pivoting of both first and second jaw members 102, 104 relative to the shaft 108. However, in other embodiments (not shown), the stabilizing member 106 may include a single slot 126 when pivoting only one of the first and second jaw members 102, 104 is desired. The slots 126 may be linearly angled relative to the longitudinal axis of the clip applier 100, such that the pins 128 simultaneously move longitudinally and vertically through the slots 126. Thus, the stabilizing member 106 retracts longitudinally while the first and second jaw members 102, 104 pivot toward each other. The slots 126 are preferably linear/straight, but can be curved to provide the simultaneous pivoting of the jaw members 102, 104 and longitudinal movement of the stabilizing member 106.

The stabilizing member 106 may be vertically and/or laterally stabilized with an extension 130. The extension 130 may be integral to the stabilizing member 106, extend proximally, and engage a longitudinal slot. 132 in the distal portion 109 of the shaft 108. The extension 130 may longitudinally slide through the longitudinal slot 132 during actuation, while preventing lateral and/or vertical play/movement of the extension 130. Thus, the stabilizing member 106 may move longitudinally relative to the pins 128 of the first and second jaw members 102, 104 without any substantial rotation or play.

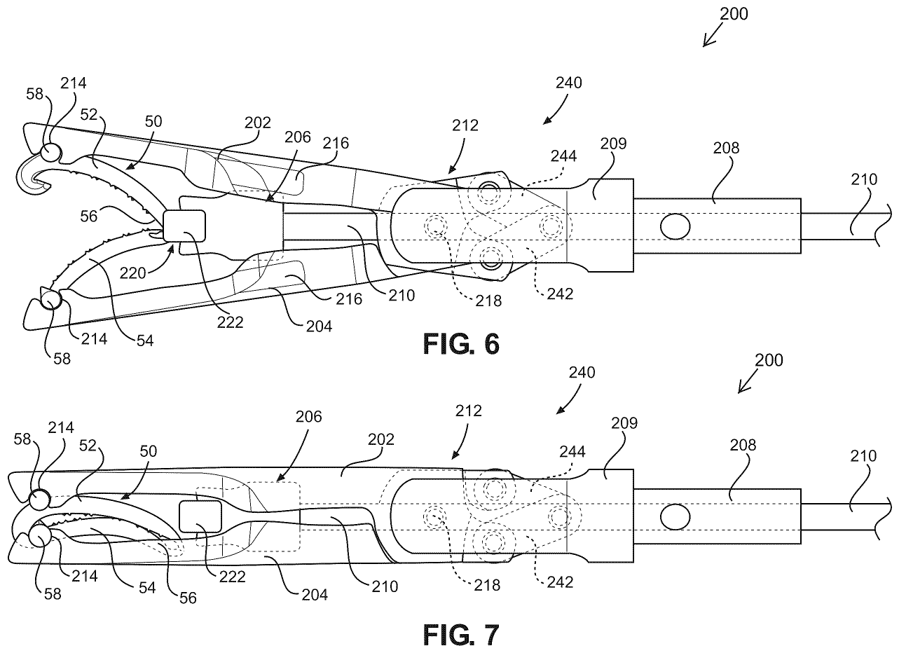

FIGS. 6-7 illustrates a second embodiment 200 of the distal end effector of the manual clip applier 10 of FIG. 1. The clip applier 200 may be configured to apply the surgical clip 50. The clip applier 200 may include a first jaw member 202, a second jaw member 204, a stabilizing member 206, a shaft 208, and a handle mechanism (as illustrated in FIG. 1). Actuation of the handle mechanism may retract and/or advance an actuating member (e.g., an actuating rod) 210 through the shaft 208 to cause the first and second jaw members 202, 204 to pivot between an open configuration (e.g., FIG. 6) and a closed configuration (e.g., FIG. 7). The first and second jaw members 202, 204 may thus compress the surgical clip 50 by pivoting the first and second leg members 52, 54 about the hinge portion 56.

The first and second jaw members 202. 204 may be pivotally coupled at a hinge mechanism 212 having a pivot pin 218 on a distal portion 209 of the shaft 208. The first and second jaw members 202, 204 may receive the surgical clip 50 between the first and second jaw members 202. 204, and the first and second jaw members 202. 204 may stabilize the surgical clip 50 at points of contact on distal portions of the first and second leg members 52, 54. For example, as similarly illustrated in FIGS. 2-5, the first and second jaw members 202, 204 may have distal portions with one or more recesses 214 that receive one or more bosses 58 on distal portions of the first and second leg members 52, 54. Each of the first and second jaw members 202. 204 may further include a longitudinal channel 216 configured to receive a portion of the surgical clip 50 and/or the stabilizing member 206.

The stabilizing member 206 may be received between the first and second jaw members 202, 204 to provide additional stability to the surgical clip 50. The stabilizing member 206 may include an elongated body having a distal portion that receives and/or engages the proximal portion (e.g., a hinge portion 16) of the surgical clip 50. The distal portion 220 may include vertical walls or protrusions 222 extending from the distal portion 220 on opposing sides of the stabilizing member 206. The vertical walls 222 may define a channel 224 therebetween configured to receive the proximal portion (e.g., the hinge portion 56) of the surgical clip 50, reducing lateral movement of the surgical clip 50. For example, the vertical walls 222 may be integrated, welded, and/or secured to opposing lateral sides of the elongated body of the stability member 206, such that the width defined by the vertical walls 222 may be wider than the width of the remaining length of the stabilizing member 206. The vertical walls 222 may be substantially parallel, and may not extend the entire height of the distal portion 220. The distal portion 220 may further include lateral protrusions (not shown) extending laterally between the vertical walls 222 to engage the proximal portion of the surgical clip 50. Thus, the distal portion 220 may receive, grip, and/or stabilize the surgical clip 50 in a lateral and/or vertical direction when positioned between the first and second jaw members 202, 204, as discussed above.

The stabilizing member 206 may be positioned symmetrically between the first and second jaw members 202, 204. The positioning of the stabilizing member 206 may allow a user to pick up the surgical clip 50 from a clip cartridge (not shown) with the clip applier 200 in either of two opposite orientations. For example, the first jaw member 202 may engage either of the first leg member 52 or the second leg member 54 of the surgical clip 50, while the second jaw member 204 engages the other of the first leg member 52 and the second leg member 54. The three point engagement between the clip applier 200 and the surgical clip 50 increases the security of the surgical clip 50. Two points of contact occur on the surgical clip 50 at distal surfaces of the first and second leg members 52, 54, and a third point of contact occurs at a proximal portion of the surgical clip 50 (e.g., the hinge portion 56). The surgical clip 50 may remain positively engaged between the first and second jaw members 202, 204, despite external forces.

As illustrated in FIGS. 6-7, the first and second jaw members 202, 204 may be configured to compress the surgical clip 50 by applying opposing forces on the first and second leg members 52. 54. The clip applier 200 may be initially loaded with the surgical clip 50 from the clip cartridge (not shown), such that the first and second jaw members 102, 104 engage distal portions of the leg members 52, 54 (e.g., with recesses 214) and the hinge portion 56 is received between the vertical walls 222 of the stabilizing member 106. Actuation of the handle mechanism may retract the actuating member 210, which may be coupled to the first and second jaw member 202, 204 through a linkage 240. For example, a first link 242 may have a first end pivotally coupled to the actuating member 210 and a second end pivotally coupled to a proximal end of the first jaw member 202. A second link 244 may have a first end pivotally coupled to the actuating member 210 and a second end pivotally coupled to a proximal end of the second jaw member 204. The attachment of the linkage 240 may be proximal of the hinge mechanism 212, such that retraction of the actuating member 210 causes the first and second jaw members 202, 204 to close.

As the first and second jaw members 202, 204 pivot, the stabilizing member 206 may be configured to move longitudinally between a first, distal position at least partially between the first and second jaw members 202, 204 (e.g., FIG. 6) and a second, proximal position at least partially between the first and second jaw members 202, 204 (e.g., FIG. 7). The stabilizing member 206 may be constrained to longitudinal movement between the first and second positions. The stabilizing member 206 may be received in the longitudinal channels 216 of the first and/or second jaw members 202, 204 in the first and/or second positions to prevent impeding of the pivoting of the jaw members 202, 204. After compression of the surgical clip 50, the jaw members 202, 204 may pivot to the open configuration thus returning stabilizing member 206 to the first, distal position to engage a second surgical clip 50 from a cartridge. The clip applier 200 may have features and function similar to the clip applier 100 except when otherwise indicated.

As illustrated in FIGS. 6-7, the movement of the stabilizing member 206 may be directly actuated by the longitudinal movement of the actuating member 210. The stabilizing member 206 may be directly attached (e.g., integral) to the actuating member 210. Thus, as illustrated in FIGS. 6-7, the actuating member 210 may extend from the handle mechanism, past the linkage 240, past the pivot pin 218, and directly attach (e.g. be integrated) to the stabilizing member 206. Retraction and advancement of the actuating member 210 would therefore cause direct retraction and advancement of the stabilizing member 206.

As further illustrated in FIGS. 4-7, the first leg member 52 of the surgical clip 50 may have a concave inner surface and a hook member on a distal portion, and the second leg member 54 may include convex inner surface and a tip member on a distal portion. As the surgical clip 50 closes, the hook on the first leg member 52 may deflect around the tip member on the second leg member to secure the surgical clip 50 in a latched configuration. Due to the curvatures, the first and/or second leg members 52, 54 may straighten and/or elongate during the closing and/or latching process. Thus, retraction of the stabilizing member 106, 206 as discussed herein prevents interference with the closing and/or latching of the surgical clip 50. The retraction of the stabilizing member 106, 206 also facilitates release of the surgical clip 50 after closing and/or latching of the surgical clip 50.

The surgical clip 50 may be made of any suitable size and may be applied to any number of tissues, such as blood vessels, lymph nodes, nerves, fallopian tubes, or cardiac tissue. The surgical clip 50 may be constructed from any suitable biocompatible material, such as metals and polymers. In some embodiments, the surgical clip 50 consists of a one-piece integral polymeric body formed from a suitable strong biocompatible engineering plastic such as the type commonly used for surgical implants. Exemplary materials include homopolymer or co-polymer polyacetal, polyethylene terephthalate (PET), polybutylene terephthalate (PBT), polyoxymethylene, or other thermoplastic materials having similar properties that can be injection-molded, extruded, or otherwise processed into like articles. Embodiments of the surgical clip 50 are further disclosed in U.S. Pat. No. 4,834,096, the disclosure of which is incorporated herein by reference. Embodiments of a cartridge containing the surgical clip 50 are further disclosed in U.S. Pat. No. 6,880,699, the disclosure of which is incorporated herein by reference.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A clip applier configured to apply a surgical clip, the clip applier comprising:
a first jaw member configured to engage the surgical clip;
a second jaw member configured to engage the surgical clip;
an actuating member configured to pivot at least one of the first and second jaw members between an open configuration and a closed configuration, the actuating member coupled to the first and second jaw members through a linkage; and
a stabilizing member attached to the actuating member, the stabilizing member being disposed between the first and second jaw members, and the stabilizing member having an elongated body including a distal portion configured to receive and/or engage a proximal portion of the surgical clip;
wherein the stabilizing member is configured to move with respect to the clip applier from a distal position at least partially between the first and second jaw members to a proximal position at least partially between the first and second jaw members,
wherein the distal portion of the stabilizing member is configured to stabilize the proximal portion of the surgical clip in a lateral direction when the first and second jaw members are in the open configuration, and
wherein advancement of the actuating member causes the stabilizing member to advance as the first and second jaw member pivot to the open configuration, and retraction of the actuating member causes the stabilizing member to retract as the first and second jaw members pivot to the closed configuration.

2. The clip applier of claim 1, wherein the linkage comprises a first link and a second link, the first link having a first end pivotally coupled to the actuating member and a second end pivotally coupled to a proximal end of the first jaw member, and the second link having a first end pivotally coupled to the actuating member and a second end pivotally coupled to a proximal end of the second jaw member.

3. The clip applier of claim 1, wherein the first and second jaw members are pivotally coupled at a hinge mechanism having a pivot pin.

4. The clip applier of claim 3, wherein the linkage is proximal of the hinge mechanism and the pivot pin.

5. The clip applier of claim 1, wherein the first jaw member includes a first longitudinal channel configured to receive a first portion of the surgical clip and/or a first portion of the stabilizing member, and the second jaw member includes a second longitudinal channel configured to receive a second portion of the surgical clip and/or a second portion of the stabilizing member.

6. The clip applier of claim 5, wherein the first portion of the stabilizing member is received in the first longitudinal channel of the first jaw member in the distal and/or proximal positions, and the second portion of the stabilizing member is received in the second longitudinal channel of the second jaw member in the distal and/or proximal positions.

7. The clip applier of claim 1, wherein the stabilizing member is constrained to longitudinal movement between the first and second positions.

8. The clip applier of claim 1, wherein the distal portion of the stabilizing member includes a pair of protrusions, each respective protrusion extending from the distal portion on opposing sides of the stabilizing member, the protrusions defining a channel therebetween configured to receive the proximal portion of the surgical clip for reducing lateral movement of the surgical clip when the first and second jaws are in the open configuration.

9. The clip applier of claim 1, wherein the stabilizing member is configured to disengage the surgical clip when the first and second jaw members are in the closed configuration.

10. The clip applier of claim 1, wherein the first jaw member is configured to engage a distal portion of a first leg member of the surgical clip, and the second jaw member is configured to engage a distal portion of a second leg member of the surgical clip.
